(19) United States
(12) Patent Application Publication
(10) Pub. No.: US 2010/0029902 A1
(43) Pub. Date: Feb. 4, 2010

(54) COATED NANOPARTICLES AND METHOD FOR COATING NANOPARTICLES

(75) Inventors: Yo-Han CHOI, Daejeon (KR); Sanghee Kim, Daejeon (KR)

Correspondence Address: AMPACC Law Group, 3500 188th Street S.W., SUITE 103, Lynnwood, WA, 98037, US

(73) Assignee: Electronics and Telecommunications Research Institute, Daejeon (KR)

(21) Appl. No.: 12/324,821

(22) Filed: Nov. 26, 2008

(30) Foreign Application Priority Data
Jul. 29, 2008 (KR) 2008-73821

(51) Int. Cl. C07K 2/00

(52) U.S. Cl. 530/300

(57) ABSTRACT

Provided are coated nanoparticles and a method for coating nanoparticles. The coated nanoparticles are coated with proteins to prevent aggregation of the coated nanoparticles. The method for coating nanoparticles may include mixing an excessive amount of proteins with the nanoparticles. Alternatively, the method for coating nanoparticles may include coating with first proteins, treating with ultrasonic waves, and coating with second proteins.

### CROSS-REFERENCE TO RELATED APPLICATIONS

This U.S. non-provisional patent application claims priority under 35 U.S.C. §119 of Korean Patent Application No. 10-2008-0073821, filed on Jul. 29, 2008, the entire contents of which are hereby incorporated by reference.

### BACKGROUND OF THE INVENTION

The present invention disclosed herein relates to nanoparticles coated with proteins and a method for coating nanoparticles using proteins.

When the size of particles is the micrometer level or less, phenomena not observed in giant particles may be found. For example, in the case of gold nanoparticles, it may be observed that the color of a solution where the gold nanoparticles are dissolved changes according to the size of the gold nanoparticles. Such a variation in a solution may be caused by the aggregation between gold nanoparticles. Based on the variation in a solution due to the aggregation between gold nanoparticles, research has been conducted into ways of applying the aggregation and the color change of gold nanoparticles depending on the presence of a specific medium, to sensors. When such nanoparticles are used, a 2-dimensional area to be used may be greatly increased. Also, a marker is immobilized to the surfaces of the nanoparticles to generate greater signals than that of the 2-dimensional area. Recently, efforts are being conducted to apply the control of the nanoparticles to biological research.

### SUMMARY OF THE INVENTION

The present invention provides coated nanoparticles having excellent characteristics.

The present invention also provides a method for coating nanoparticles having excellent characteristics through simple processes.

Embodiments of the present invention provide nanoparticle compositions including: nanoparticles; and one or more types of proteins adsorbed to an entire surface of the respective nanoparticles to control aggregation of the nanoparticles.

In some embodiments, the nanoparticle may deliver the proteins to a target material, and at least one type of protein among the proteins may react with the target material.

In other embodiments, the proteins may include first proteins and second proteins, and the first proteins may have reactivity with the target material, and the second proteins may have no reactivity with the target material.

In still other embodiments, the proteins may include first proteins and second proteins, and the first proteins may have reactivity with the target material, and the second proteins may have different reactivity from that of the first proteins with the target material.

In even other embodiments, the second proteins may have a larger amount than the first proteins.

In yet other embodiments, the nanoparticle may be an organic material.

In other embodiments of the present invention, methods for coating nanoparticles include: mixing the nanoparticles and proteins with a dispersed solution, wherein the proteins are adsorbed to entire surfaces of the nanoparticles, control aggregation of the nanoparticles, and include at least one protein type.

In some embodiments, the mixing of the nanoparticles and the proteins with the dispersed solution may include: providing the proteins with an amount larger than an amount adsorbable to the entire surfaces of the nanoparticles.

In other embodiments, the proteins may include first proteins and second proteins, and the mixing of the nanoparticles and the proteins with the dispersed solution may include: dispersing the nanoparticles into the dispersed solution; primarily mixing the first proteins with the dispersed solution; and treating the dispersed solution with ultrasonic waves.

In still other embodiments, the methods may further include secondarily mixing the second proteins with the dispersed solution.

In even other embodiments, the secondarily mixing of the second proteins with the dispersed solution may include mixing the second proteins in a larger amount than that of the first proteins.

### BRIEF DESCRIPTION OF THE FIGURES

The accompanying figures are included to provide a further understanding of the present invention, and are incorporated in and constitute a part of this specification. The drawings illustrate exemplary embodiments of the present invention and, together with the description, serve to explain principles of the present invention. In the figures:

FIG. 1 is a schematic view illustrating aggregation patterns of nanoparticles according to exemplary embodiments of the present invention with comparison examples;

FIGS. 2A and 2B are scanning electron microscophy (SEM) images illustrating nanoparticles according to a first comparison example of the present invention;

FIGS. 2C and 2D are SEM images illustrating nanoparticles according to a second comparison example of the present invention;

FIGS. 2E and 2F are SEM images illustrating nanoparticles according to an exemplary embodiment of the present invention;

FIG. 3 is a graph illustrating optical density of nanoparticles according to embodiments of the present invention with comparison examples; and

FIG. 4 is a graph illustrating adsorption extent of proteins according to embodiments of the present invention.

### DETAILED DESCRIPTION OF PREFERRED EMBODIMENTS

Hereinafter, it will be described about an exemplary embodiment of the present invention in conjunction with the accompanying drawings.

Preferred embodiments of the present invention will be described below in more detail with reference to the accompanying drawings. The present invention may, however, be embodied in different forms and should not be constructed as limited to the embodiments set forth herein. Rather, these embodiments are provided so that this disclosure will be thorough and complete, and will fully convey the scope of the present invention to those skilled in the art. Like reference numerals refer to like elements throughout.

In the figures, the dimensions of layers and regions are exaggerated for clarity of illustration. It will also be understood that when a layer (or film) is referred to as being ‘on’ another layer or substrate, it can be directly on the other layer or substrate, or intervening layers may also be present. Further, it will be understood that when a layer is referred to as being ‘under’ another layer, it can be directly under, and one or more intervening layers may also be present. In addition, it will also be understood that when a layer is referred to as being ‘between’ two layers, it can be the only layer between the two layers, or one or more intervening layers may also be present.

It will also be understood that the term ‘and/or’ is used herein to describe any one or all of elements located at the front and the rear of this term.

[Coated Nanoparticles]

A coated nanoparticle may include a nanoparticle and proteins adsorbed to the entire surface of the nanoparticle according to an examplary embodiment of the present invention. For example, the nanoparticle may include polystyrene as an organic material. The nanoparticle may deliver the proteins to a target material (a target molecule or an analyte), and the proteins may react with the target material. For example, the protein may be an immune body or an enzyme such as an immunoglobulin G (IgG). The target material may be a target to be reacted with the protein or a target to be analyzed with the protein.

According to an examplary embodiment of the present invention, all of the proteins may react with the target material. According to another examplary embodiment of the present invention, a part of the proteins may react with the target material.

For example, the proteins may include first proteins and second proteins. While the first proteins may have reactivity with the target material, the second proteins may be non-reactive with the target material. Alternately, the second proteins and the first proteins may have different reactivity with the target material, respectively. For example, when the first protein is an immune body, the second protein may be a bovine serum albumin (BSA). That is, the second proteins may be adsorbed to the surfaces of the nanoparticles to which the first protein is not adsorbed and block the surfaces of the nanoparticles. Here, the first proteins may be used in a smaller amount than the second proteins.

[Coating Method 1]

Hereinafter, a method of coating nanoparticles will be described according to an examplary embodiment of the present invention.

A dispersed solution is prepared. The dispersed solution may be determined depending on a mixed material and be, e.g., a phosphate-buffered saline. The nanoparticles may be mixed with the dispersed solution. For example, the nanoparticle may be an organic material such as polystyrene. Proteins may be mixed with a mixture of the nanoparticles and the dispersed solution. For example, the protein may be an immune body or an enzyme. The proteins may be provided in an excessive amount and be adsorbed to the entire surfaces of the nanoparticles. For example, a mixed amount of the protein may be equal to or greater than an amount with which the proteins are adsorbed to the entire surfaces of the nanoparticles. Accordingly, the surfaces of the nanoparticles may be coated with the proteins.

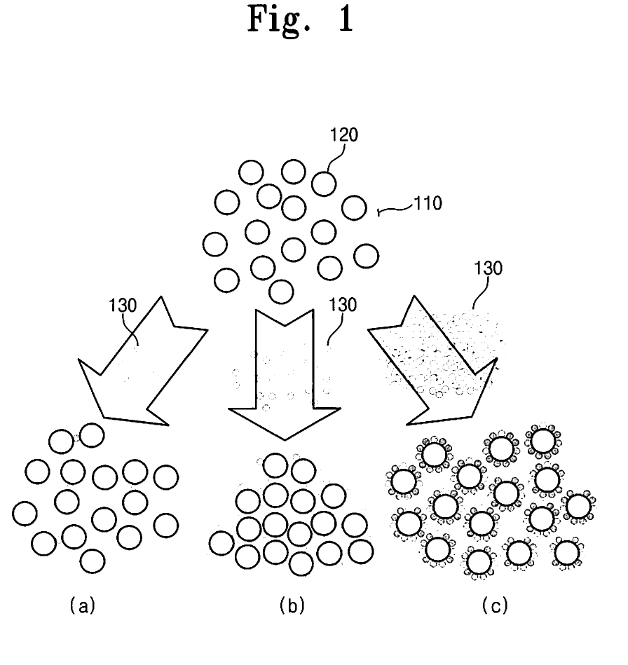

Referring to FIG. 1, a method of coating nanoparticles according to an exemplary embodiment of the present invention is described with comparison examples.

According to the first comparison example of the present invention, a small amount of proteins 130 may be mixed with a dispersed solution 110 including nanoparticles 120 (‘a’ of FIG. 1). For example, a mixed amount of the proteins 130 with about 1 ml of the dispersed solution 110 including about 1 mg of the nanoparticles 120 having an average diameter of about 110 nm, may range from about 0.1 μg to about 1 μg. The protein 130 adsorbed to one of the nanoparticles 120 may be simultaneously adsorbed to the adjacent nanoparticle 120. Thus, the nanoparticles 120 may be partially aggregated by the proteins 130 in the dispersed solution 110.

According to the second comparison example of the present invention, an excessive amount of the proteins 130 relative to ‘a’ of FIG. 1 may be mixed with the dispersed solution 110 including the nanoparticles 120 (‘b’ of FIG. 1). A mixed amount of the proteins 130 is less than an amount with which the proteins 130 cover the entire surfaces of the nanoparticles 120. For example, a mixed amount of the proteins 130 with about 1 ml of the dispersed solution 110 including about 1 mg of the nanoparticles 120, may range from about 10 μg to about 100 μg. The protein 130 adsorbed to one of the nanoparticles 120 may be simultaneously adsorbed to the plurality of adjacent nanoparticles 120. Thus, the nanoparticles 120 may be extensively aggregated by the proteins 130 in the dispersed solution 110. For example, in ‘a’ of FIG. 1, when one of the proteins 130 is adsorbed to one of the nanoparticles 120, the protein 130 may aggregate the two adjacent nanoparticles 120. On the other hand, as illustrated in ‘b’ of FIG. 1, when a plurality of proteins 130 are adsorbed to the single nanoparticle 120, the proteins 130 may aggregate three or more adjacent nanoparticles 120. When the nanoparticles 120 are aggregated in this manner, the proteins 130 adsorbed to the nanoparticles 120 may lose their original characteristics. For example, the protein 130 may lose its function as an immune body or an enzyme.

According to an examplary embodiment of the present invention, an excessive amount of the proteins 130 relative to b’ of FIG. 1 may be mixed with the dispersed solution 110 including the nanoparticles 120 (‘c’ of FIG. 1). The proteins 130 may be adsorbed to the entire surface of the respective nanoparticles 120. Here, a mixed amount of the proteins 130 may be larger than an amount with which the proteins 130 cover the entire surfaces of the nanoparticles 120. For example, a mixed amount of the proteins 130 with about 1 ml of the dispersed solution 110 including about 1 mg of the nanoparticles 120, may be about 1 mg or more. Accordingly, the entire surfaces of the nanoparticles 120 are coated with the proteins 130 so that the aggregation of the adjacent nanoparticles 120 is prevented. The aggregation between or among the nanoparticles 120 may happen when one protein 130 is adsorbed to a plurality of the nanoparticles 120 at the same time. Thus, in the case where the proteins 130 are adsorbed to the entire surfaces of the nanoparticle 120, the nanoparticles 120 have no remaining surfaces allowing the proteins 130 to be adsorbed to the adjacent nanoparticles 120. As a result, the nanoparticles 120 are dispersed without the aggregation.

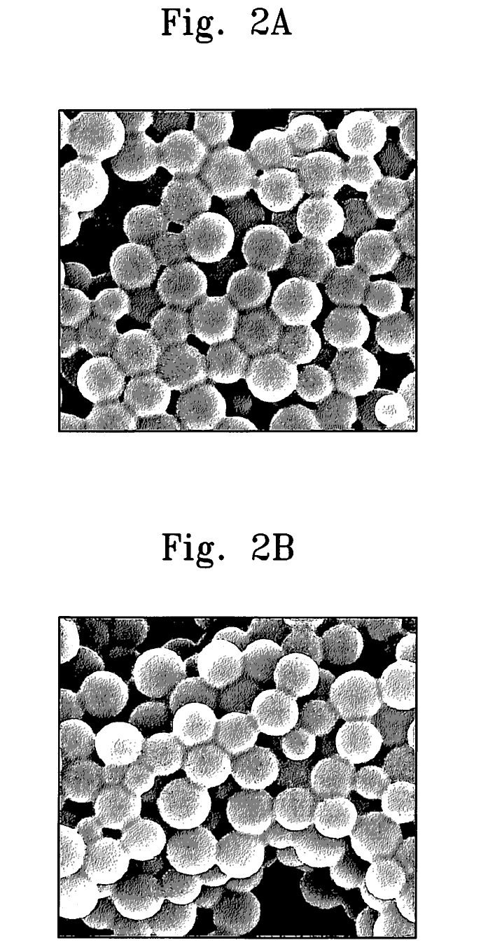

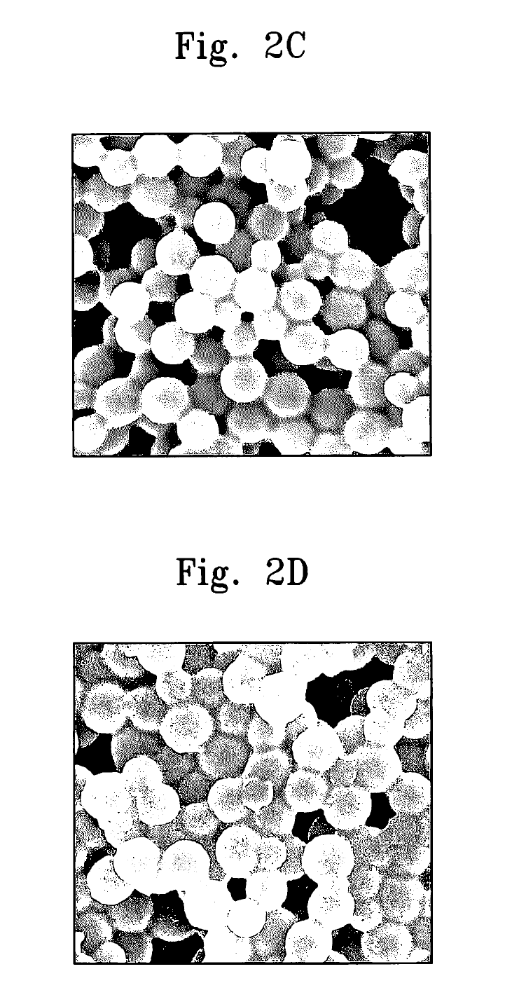

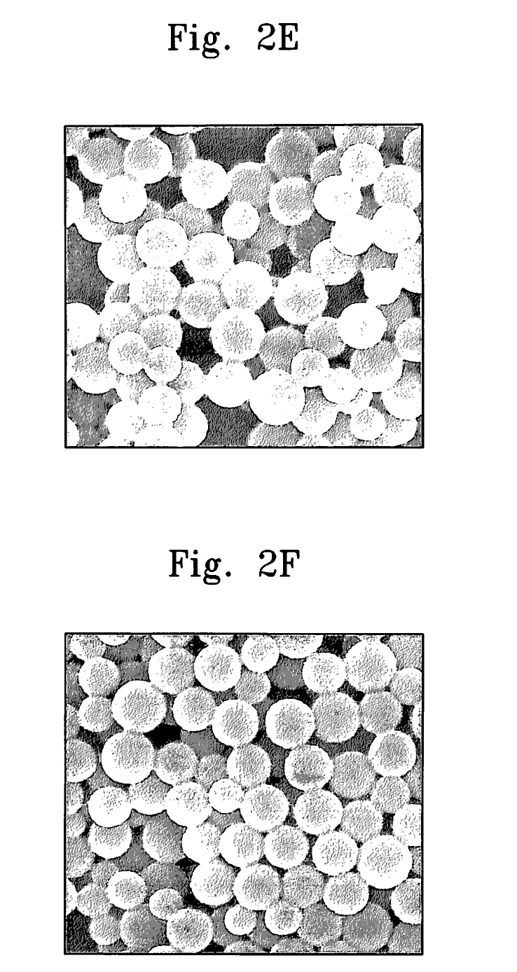

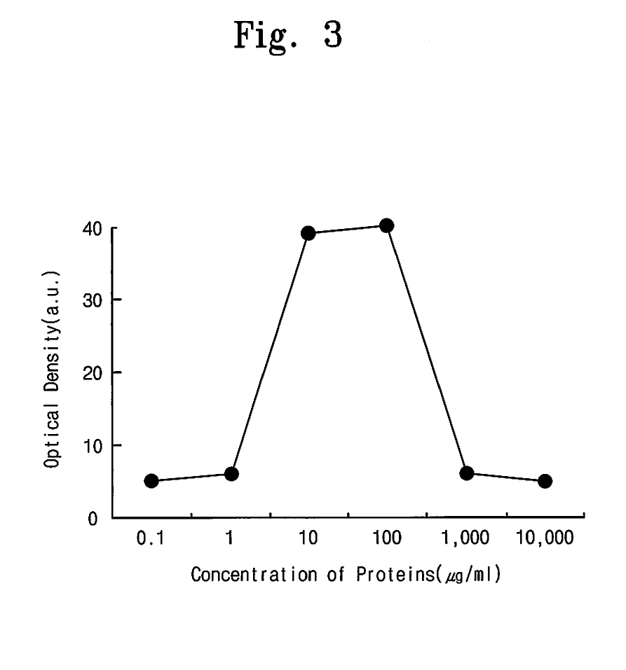

Hereinafter, referring to FIGS. 2A through 2F, and FIG. 3, aggregation characteristics of the nanoparticles according to the examplary embodiment with the comparison examples will now be described. FIGS. 2A through 2F are SEM images illustrating the coated nanoparticles by supplying about 0.1 μg of the proteins (FIG. 2A), about 1 μg of the proteins (FIG. 2B), about 10 μg of the proteins (FIG. 2C), about 100 μg of the proteins (FIG. 2D), about 1 mg of the proteins (FIG. 2E), and about 10 mg of the proteins (FIG. 2F), respectively.

Referring to FIGS. 2A and 2B, in the case of the nanoparticles treated with a small amount of the proteins, aggregation was not observed with naked eyes. In only the SEM image, some aggregation was partially observed at contact surfaces between the nanoparticles.

Referring to FIG. 2C and 2D, in the case of the nanoparticles treated with a larger amount of the proteins, aggregation was observed with naked eyes. In the SEM image, extensive aggregation was observed.

Referring to FIG. 2E and 2F, in the case of the nanoparticles treated with an excessive amount of the proteins, so that the entire surfaces of the proteins are coated, aggregation was not observed with naked eyes. Also, the SEM images showed that the nanoparticles separately existed without aggregation. The SEM images also showed that the nanoparticles of the FIGS. 2E and 2F were spheres with more improved morphology than the nanoparticles of the FIGS. 2A and 2B.

FIG. 3 illustrates optical density (OD) (or absorbance) of a mixture according to a mixed amount (or concentration) of proteins. To measure the optical density, a light having a wavelength of about 600 nm was irradiated to a mixture including nanoparticles treated with proteins. Then, the intensity of the light transmitting through the mixture was measured. As transmittance becomes low, the OD becomes high, thus as the OD becomes high, aggregation rate of the nanoparticles (or amount of the aggregated nanoparticles) becomes high.

As illustrated in FIGS. 2A and 2B, a mixture treated with a small amount (e.g., several μg or less) of proteins had a low OD. As illustrated in FIGS. 2C and 2D, as a mixed amount of proteins was increased, the OD of the mixture was increased. However, as illustrated in FIGS. 2E and 2F, the OD of a mixture treated with an excessive amount of proteins was quickly decreased. Accordingly, it has been found that when a mixed amount of proteins is very small relative to nanoparticles and when a mixed amount of proteins is sufficiently large to coat the entire surfaces of the nanoparticles, the nanoparticles are not aggregated.

[Coating Method 2]

Hereinafter, a method of coating nanoparticles will now be described according to another embodiment of the present invention.

A dispersed solution is prepared. Nanoparticles may be mixed with the dispersed solution. For example, the nanoparticles may be organic materials. Primarily, first proteins may be mixed with a mixture of the nanoparticles and the dispersed solution. The nanoparticle may deliver the first proteins to a target material, and the proteins may react with the target material. For example, the first protein may be an immune body or an enzyme. The first proteins may be provided with an amount which is less than an amount for coating the entire surfaces of the nanoparticles, and which is simultaneously causing the aggregation of the nanoparticles. Thus, the nanoparticles may be partially aggregated.

Secondarily, second proteins are further mixed with the mixture. The second proteins may be non-reactive with the target material. Alternatively, the second proteins may have different reactivity from that of the first proteins. An amount of the further mixed second proteins may be equal to or greater than an amount for covering the surfaces of the remaining nanoparticles that the primarily provided first proteins do not cover.

After that, an ultrasonic treating process may be performed on the mixture such that the aggregated nanoparticles are dispersed in the mixture. At this point, the further mixed second proteins may be adsorbed to the remaining surfaces of the dispersed nanoparticles. Accordingly, the entire surfaces of the nanoparticles may be coated with the proteins. The second proteins are adsorbed to the surfaces of the nanoparticle to which the first proteins are not adsorbed and thus the second proteins may block the surfaces of the nanoparticles.

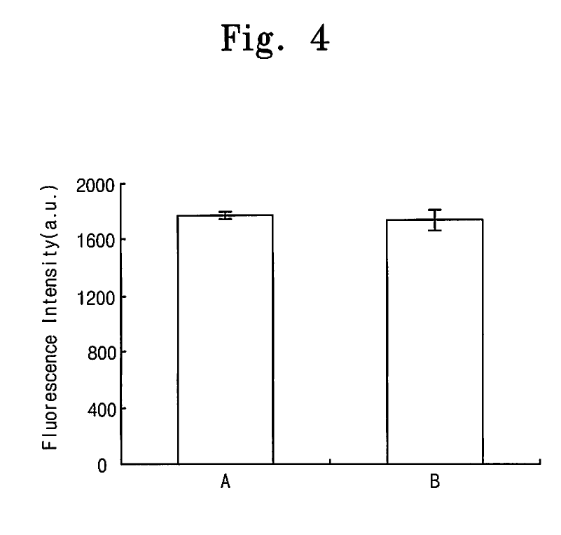

Referring to FIG. 4, adsorption strength (i.e., adsorption or desorption extent) of the first proteins through the ultrasonic treating process will now be described according to an embodiment of the present invention.

A mixture solution including about 1 ml of phosphate-buffered saline (PBS) with about 1 mg of polystyrene nanoparticles and about 10 μg of the first proteins labeled with a fluorescent material was prepared. A fluorescein isothiocyanate (FITC) was used as the fluorescent material. The aggregation of the nanoparticles occurred in the mixture solution. The mixture solution was centrifuged in a centrifuge with about 28500 g (where g is gravitational acceleration) for about 30 minutes. The nanoparticles of the mixture solution go down by the centrifuging, and a supernatant was removed.

Then, a mixture solution of the nanoparticles and about 1 ml of new PBS was formed. Thereafter, the mixture solution was also centrifuged. After the centrifuging process was repeated 5 times , the mixture solution mixed with about 1 mg of the PBS was formed.

The mixture solution formed through the processes was provided as a first test solution B and a second test solution A. The second solution A was used for a test, without a treatment, but the first test solution B was treated as described below. About 10 mg of the second proteins, without being labeled with a fluorescent material, was added to the first test solution B and then the ultrasonic treating process was performed as the previous embodiment. Then a washing process with the centrifuging was performed about 5 times .

Samples with the same amount are taken from the first test solution B and the second test solution A, respectively. Fluorescence of the samples of the first test solution B and the second test solution A was measured using a fluorometer (SpectraMax M2, molecular devices). Referring to FIG. 4, the first test solution B had the similar fluorescence as the second test solution A. The fluorescence due to the amount of the fluorescent material represents a relative amount of the first proteins labeled with the fluorescent material. The fluorescence of the first test solution B was decreased by about 2% or less, comparing with the second test solution A. Thus, according to the embodiment, although the ultrasonic treating process is performed, the previously adsorbed first proteins to the nanoparticles are not desorbed. Thus, the nanoparticles are separated without desorption of the previously adsorbed proteins.

According to the present invention, the nanoparticles are coated with proteins to prevent the aggregation between the nanoparticles. Thus, nanoparticles having an excellent dispersed performance can be provided. The nanoparticles can be coated with mixed proteins having a larger amount than a necessary amount for coating the surface of the nanoparticles. Alternatively, the nanoparticles may be mixed with a small amount of reactive proteins, and then mixed with an excessive amount of non-reactive proteins, and treated with ultrasonic waves. Accordingly, it is possible to prevent the aggregation between the nanoparticles with an economical method.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

1. A nanoparticle composition comprising:
nanoparticles; and
one or more types of proteins that are adsorbed on an entire surface of the respective nanoparticles, where the nanoparticles have substantially no remaining surfaces for the proteins to be adsorbed thereon, wherein the proteins adsorbed on the entire surface of the nanoparticles enable dispersion of the nanoparticles without aggregation.

2. The nanoparticle composition of claim 1, wherein the nanoparticles is suitable for delivering the proteins to a target material, and at least one type of protein among the proteins has suitable characteristics for reacting with the target material.

3. The nanoparticle composition of claim 2, wherein the proteins comprise first proteins and second proteins, and
the first proteins have reactivity with the target material, and the second proteins have no reactivity with the target material.

4. The nanoparticle composition of claim 2, wherein the proteins comprise first proteins and second proteins, and
the first proteins have reactivity with the target material, and the second proteins have different reactivity from that of the first proteins with the target material.

5. The nanoparticle composition of claim 3, wherein the one or more types of proteins comprise a greater amount of the second proteins than the first proteins.

6. The nanoparticle composition of claim 2, wherein the nanoparticles are an organic material.

7. A method for coating nanoparticles, the method comprising:
mixing the nanoparticles and proteins with a dispersed solution,
wherein the proteins are adsorbed to entire surfaces of the nanoparticles, control aggregation of the nanoparticles, and include one or more protein types.

8. The method of claim 7, wherein mixing the nanoparticles and the proteins with the dispersed solution comprises:
providing the proteins with an amount larger than an amount adsorbable to the entire surfaces of the nanoparticles.

9. The method of claim 8, wherein the proteins comprise first proteins and second proteins, and
mixing the nanoparticles and the proteins with the dispersed solution comprises:
dispersing the nanoparticles into the dispersed solution;
primarily mixing the first proteins with the dispersed solution; and
treating the dispersed solution with ultrasonic waves.

10. The method of claim 9, further comprising secondarily mixing the second proteins with the dispersed solution.

11. The method of claim 10, wherein secondarily mixing of the second proteins with the dispersed solution comprises mixing the second proteins in a larger amount than that of the first proteins.

12. A nanoparticle composition comprising:
nanoparticles; and
first and second proteins that are adsorbed on an entire surface of the respective nanoparticles, where the nanoparticles have substantially no remaining surfaces for the first and second proteins to be adsorbed thereon, wherein the proteins adsorbed on the entire surface; of
the nanoparticles enable dispersion of the nanoparticles without aggregation, wherein the first proteins have reactivity with a target material, and the second proteins have no reactivity with the target material, wherein an amount of the second proteins is greater than an amount of the first protein on the surface of the respective nanoparticles.
